(19) United States
(12) Patent Application Publication
(10) Pub. No.: US 2008/0176963 A1
(43) Pub. Date: Jul. 24, 2008

(54) TREATMENT OF BRAIN DISORDERS OF THE STRIATUM

(76) Inventors: Robert C. Malenka, Palo Alto, CA (US); Anatol Kreitzer, Palo Alto, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/018,063

(22) Filed: Jan. 22, 2008

Related U.S. Application Data
(60) Provisional application No. 60/897,322, filed on Jan. 24, 2007.

(51) Int. Cl. A61K 35/00; A61P 25/16

(52) U.S. Cl. 514/789

(57) ABSTRACT

Regulation of striatal indirect pathway endocannabinoid-mediated long-term depression (eCB-LTD) is used to improve motor deficits of striatal-based brain disorders. In such treatment, a dopamine D2 receptor agonist is administered in conjunction with an inhibitor of endocannabinoid degradation. In some embodiments, the inhibitor of endocannabinoid degradation is a fatty acid amine hydrolase inhibitor, or a monoacylglycerol lipase antagonist. The combination provides for a synergistic effect, with improved therapeutic effects. Also provided are kits and systems for practicing the subject methods, as well as methods of use of agents identified in the screening method of the invention.

### BACKGROUND OF THE INVENTION

Endocannabinoids are a class of lipophilic membrane-derived signaling molecules, the most prominent of which are anandamide and 2-arachidonoylglycerol (2-AG). Both depolarization and metabotropic receptor activation can elicit endocannabinoid release. Endocannabinoids may bind to one or both of the CB1 and CB2 receptors.

Endocannabinoids are retrograde transmitters that most commonly travel backwards against the usual synaptic transmitter flow, often being released from the postsynaptic cell and acting on the presynaptic cell. Activation of cannabinoid receptors temporarily reduces the amount of conventional neurotransmitter released. Postsynaptic endocannabinoid release provides a mechanism for rapid retrograde regulation of synaptic strength on both short and long time scales. This endocannabinoid mediated system permits the postsynaptic cell to control its own incoming synaptic traffic. Endocannabinoids thus constitute a versatile system for affecting neuronal networks.

The striatum is the point of entry of information into the basal ganglia, and it has important roles in motor control and habit learning. The neocortex and thalamus provide the major excitatory inputs to striatal medium spiny projection neurons (MSNs). Morphological studies have demonstrated that the majority of these afferent terminals impinge on the head of the spines on the dendrites of striatal MSNs, whereas most dopaminergic afferent fibers coming from the substantia nigra make synapses on the necks of the same dendritic spines. This close anatomical localization of these two types of synapses has suggested that dopamine (DA) released from the nigrostriatal afferent terminals may have modulatory effects on the excitatory signals generated from the cortex and thalamus. The importance of DA in normal striatal function is evidenced by the severe disruption of behavior observed in Parkinson's disease and after chemical lesions of nigral dopaminergic inputs to striatum. In fact, DA plays a variety of important physiological roles in striatum.

Two important properties of medium spiny neurons that distinguish them from other cell types are their down-state to up-state transitions and their rich innervation by midbrain dopaminergic afferents. In vivo, medium spiny neurons rest in the down state, at resting membrane potentials ranging between −60 and −90 mV. Synchronous excitatory input from cortex shifts the membrane potential of medium spiny neurons cells by 20-30 mV to the up state, which ranges between −40 and −70 mV. These state transitions affect both the source and magnitude of postsynaptic calcium signaling. The control of striatal synaptic plasticity by DA is particularly important, given that influential theories of reinforcement learning require such DA-dependent modulation.

Mechanisms controlling endocannabinoid release from medium spiny neurons also differ from other cell types. Unlike neurons in other brain regions, strong depolarization alone is not sufficient to trigger endocannabinoid release. Instead, mGluR activation combined with L-type calcium channel activation is required and triggers eCB-LTD, which can be generated by pairing moderate-frequency afferent stimulation with brief depolarizations that mimic up-state transitions. This eCB-LTD is prevented by blocking DA D2 receptors and enhanced by their activation. Thus, the endocannabinoid release from medium spiny neurons that triggers LTD in the dorsal striatum requires both DA release and up-state-dependent calcium signaling.

Both D₁ and D₂ receptors can modulate multiple voltage-dependent conductances in medium spiny neurons in complex ways. Studies have suggested that both D₁ and D₂ receptors may be important for LTD, whereas D₁ receptors may be required for long-term potentiation. Endogenous DA importantly contributes to the generation of eCB-LTD by synaptic activity, and D₂ receptor activation enhances endocannabinoid release in the striatum in vivo. Release of DA in the striatum has specifically been suggested to gate long-term synaptic plasticity. Consistent with this idea, DA receptor activation has been reported to be required for various forms of synaptic plasticity in the striatum. Enhancement of endocannabinoid release by DA is critical for eliciting eCB-LTD.

Signalling at CB1 cannabinoid receptors plays a key role in the control of movement in health and disease. In recent years, an increased understanding of the physiological role of transmission at CB1 receptors throughout the basal ganglia circuitry has led to the identification of therapeutic approaches to both the symptoms of Parkinson's disease and the side effects of current anti-parkinsonian therapies. However, the actual role of endocannabinoids remains uncertain. It has been suggested that both CB1 cannabinoid receptor antagonists and agonists can modulate the behavioural effects of L-dopa. For example Silverdale et al. (2001) Exp Neurol. 2001 169(2):400-6 suggest that cannabinoid receptor antagonists may provide a useful treatment for the symptoms of Parkinson's disease. Segovia et al. (2003) Mov Disord. 18(2):138-49 found that a CB1 cannabinoid receptor antagonist reduced the increase in vertical activity elicited by L-dopa; a CB1 cannabinoid receptor agonist also reduced the L-dopa-induced increase in vertical activity; and an inhibitor of endocannabinoid transport had no effect on horizontal or vertical activity. In contrast, Ferrer et al. (2003) Eur J. Neurosci. 18(6):1607-14 suggest that a deficiency in endocannabinoid transmission may contribute to levodopa-induced dyskinesias and that these complications may be alleviated by activation of CB1 receptors.

The role of endocannabinoids on striatal signaling is of great clinical interest. In particular, imbalances between neural activity in direct pathway (striatal projection neurons targeting the substantia nigra pars reticulate) and the indirect pathway (striatal projection neurons targeting the lateral globus pallidus) have been proposed to underlie the profound motor deficits observed in Parkinson's disease and Huntington's disease. The present invention addresses this issue.

### SUMMARY OF THE INVENTION

Compositions and methods are provided for the treatment of striatal-based brain disorders. Disorders of interest include Parkinson's disease and Huntington's disease. It is shown herein that indirect pathway endocannabinoid-mediated long-term depression (eCB-LTD), which requires dopamine D2 receptor activation, is absent in models for Parkinson's disease. The coadministration of a D2 receptor agonist and an inhibitor of endocannabinoid degradation rescues this eCB-LTD and reduces the associated motor deficits.

In one embodiment of the invention, motor deficits associated with a striatal-based brain disorder in a patient are treated by the co-administration of a D2 receptor agonist and an inhibitor of endocannabinoid degradation. In some embodiments, the inhibitor of endocannabinoid degradation is a fatty acid amine hydrolase (FAAH). In other embodiments, the inhibitor of endocannabinoid degradation is a monoacylglycerol lipase antagonist. The combination provides for a synergistic effect, with improved therapeutic effects, which may lower adverse side effects. In some embodiments, the striatal-based brain disorder is Parkinson's disease.

The active compounds used in the methods of the invention may be formulated separately or together. In one embodiment, the invention provides pharmaceutical formulations comprising an effective dose of a D2 receptor agonist, an effective dose of an inhibitor of endocannabinoid degradation, and a pharmaceutically acceptable carrier.

In another embodiment of the invention a method of identifying compounds for the treatment of brain disorders of the striatum is provided, where the effectiveness of a candidate compound on indirect pathway eCB-LTD is assessed in vitro.

These and other aspects and embodiments of the invention and methods for making and using the invention are described in more detail in the description of the drawings and the invention, the examples, the claims, and the drawings that follow.

### BRIEF DESCRIPTION OF THE DRAWINGS

FIG. 1. Presynaptic properties of direct-pathway and indirect-pathway synapses on striatal MSNs. a) Sagittal diagram of mouse brain showing cortex (Ctx), striatum (Str), lateral globus pallidus (LGP), thalamus (Th) and substantia nigra pars reticulata (SNr). b) Composite confocal image of GFP fluorescence in a sagittal section from an M4-GFP BAC-transgenic mouse. c) Composite confocal image of GFP fluorescence in a sagittal section from a D2-GFP BAC-transgenic mouse. d) Representative recording from a GFP-positive MSN in an M4-GFP mouse. PPRs (EPSC₂/EPSC₁) are plotted against interstimulus interval (ISI). Points represent averages of three or four trials (filled circles). Inset: traces from an individual trial. e) Summary graph of PPRs from GFP-positive (direct pathway, filled circles; n=11) and GFP-negative (indirect pathway, open circles; n=7) neurons in M4-GFP mice plotted against ISI. f) Representative recording from a GFP-positive MSN in a D2-GFP mouse. PPRs are plotted against ISI. Points represent averages of three or four trials (filled circles). Inset: traces from an individual trial. g) Summary graph of PPRs from GFP-positive (indirect, open circles; n=9) and GFP-negative (direct pathway, filled circles; n=10) neurons in D2-GFP mice, plotted against ISI. h) Sample traces of mEPSCs recorded in tetrodotoxin (1 μM) from direct-pathway and indirect-pathway neurons. i) Cumulative probability plots for mEPSC inter-event interval in direct pathway (thick trace; n=4) and indirect-pathway (thin trace; n=5) neurons. Inset: mean frequency for direct-pathway (D) and indirect pathway (I) neurons. j) Cumulative probability plot for mEPSC amplitude in direct-pathway (thick trace) and indirect-pathway (thin trace) neurons. Inset: mean amplitude for direct (D) and indirect (I) pathways. Asterisk, P<0.05. Error bars indicate s.e.m.

FIG. 2. Postsynaptic properties of direct-pathway and indirect-pathway synapses. a) Summary of normalized EPSC amplitudes recorded in APV (50 μM) with spermine (0.1 mM) in the pipette at different holding potentials in direct-pathway (filled circles; n=4) and indirect-pathway (open circles; n=5) MSNs. b) EPSC traces recorded at −60 mV and 140 mV from a representative direct-pathway MSN. c) EPSC traces recorded at −60 mV and 140 mV from a representative indirect-pathway MSN. d) Summary of the ratio of the NMDA-receptor EPSC (measured at 50 ms after stimulus, holding potential: −40 mV) to the AMPA-receptor EPSC (measured at peak, holding potential +60 mV) at direct-pathway (n=14) and indirect-pathway (n=14) synapses. e) f) Current-clamp recording from a representative direct-pathway (e) or indirect-pathway (f) MSN displaying responses to injected current (50-pA steps). g) Summary of firing frequency in response to injected current in direct-pathway (filled circles; n=7) and indirect-pathway (open circles; n=8) neurons. h) Summary of normalized interspike interval (ISI) plotted as a function of spike number. Filled circles, direct; open circles, indirect. Asterisk, P<0.05. Error bars indicate s.e.m.

FIG. 3. Endocannabinoid-mediated LTD is restricted to indirect pathway synapses. a) LTD is present in indirect-pathway (open circles; n=6) but not direct-pathway (filled circles; n=12) MSNs. In this and subsequent panels, normalized EPSCs recorded from direct-pathway and indirect-pathway MSNs are plotted over time. The arrow indicates 1 s of 100 Hz stimulation, paired with postsynaptic depolarization to 0 mV, repeated four times at 10-s intervals. b) Average traces from a representative LTD experiment in a direct (top) and indirect (bottom) pathway MSN. Thick traces represent the average from 0-10 min, and thin traces represent the average from 30-40 min. c) LTD in indirect-pathway neurons is blocked by sulpiride (10 μM) (circles; n=6) and AM251 (1 μM) (triangles; n=5) but not by a combination of methysergide (4 μM), GR125487 (1 μM), LY278584 (1 μM), prazosin (2 μM) and propranolol (1 μM) (squares; n=5). d) Activation of CB1 receptors inhibits neurotransmitter release at both direct-pathway (filled circles; n=4) and indirect-pathway (open circles; n=4) synapses. WIN55, 212-2 (1 μM) and AM251 (5 μM) were applied during the time marked by the black bars. e) Application of DHPG (100 μM) for 10 min elicits LTD in indirect-pathway (open circles; n=5) but not direct-pathway (filled circles; n=6) MSNs. f) Application of DHPG (100 μM) for 1 min does not elicit LTD at indirect-pathway synapses (circles; n=5), but does elicit LTD in the presence of quinpirole (10 μM) (triangles; n=6). Error bars indicate s.e.m.

FIG. 4. Pharmacological rescue of indirect-pathway LTD and motor deficits in animal models of Parkinson's disease. a, b, LTD is absent (filled circles) in reserpine-treated mice (n=6) (a) or 6-OHDA-treated mice (n=7) (b), but is partly rescued when quinpirole (10 μM) (open circles) is present during the induction protocol in reserpine-treated mice (n=5) (a) or 6-OHDA-treated mice (n=4) (b). c, d, LTD is rescued in reserpine-treated mice (n=5) (c) or 6-OHDA-treated mice (n=4) (d) in the presence of URB597 (1 μM). Normalized EPSCs recorded from indirect-pathway MSNs are plotted over time. The arrow above the graphs in a-d indicates the time of the LTD induction protocol. e, f, Descent latency in the catalepsy bar test for reserpine-treated mice (e) and 6-OHDA-treated mice (f) before subsequent drug treatment (reserpine, n=14; 6-OHDA, n=20), and after injection with URB597 (1 mg kg⁻¹ i.p.; reserpine, n=4; 6-OHDA, n=5), quinpirole (1.5 mg kg⁻¹ i.p.; reserpine, n=5; 6-OHDA, n=10), or URB597 and quinpirole together (reserpine, n=7; 6-OHDA, n=5). g, h, Locomotor activity in the open-field test for animals treated with reserpine (g) or 6-OHDA (h) plotted as distance traveled during a 15-min test period before subsequent drug treatment (reserpine, n=19; 6-OHDA, n=20), and for mice 45-60 min after injection with URB597 (1 mg kg⁻¹ i.p.; reserpine, n=4; 6-OHDA, n=5), quinpirole (1.5 mg kg⁻¹ i.p.; reserpine, n=8; 6-OHDA, n=5), or URB597 and quinpirole together (reserpine, n=7; 6-OHDA, n=5). Asterisk, P<0.05 by one-way analysis of variance with Tukey's Honestly Significant Difference or Dunnett's test. Error bars indicate s.e.m.

FIG. 5. Muscarinic M1 receptors do not influence endocannabinoid release or eCB-LTD. a, Summary of normalized EPSC amplitudes recorded at a holding potential of −70 mV (closed circles) or −50 mV (open circles). Pirenzepine (10 μM) was applied during the time marked by the bar. b, Indirect pathway LTD is absent in the presence of sulpiride (10 μM) and pirenzepine (10 μM) (closed circles). Indirect pathway LTD in sulpiride alone (open circles) is shown for comparison (same plot as FIG. 3B). The arrow marks the time of LTD induction. The activity of pirenzepine was confirmed by its ability to block a carbachol-induced inward membrane current in MSNs (n=3, data not shown). Error bars are ±SEM.

FIG. 6. Normal presynaptic inhibition by cannabinoids in dopamine-depleted mice. a, Summary graph of paired-pulse ratios (PPR) recorded from indirect pathway neurons plotted vs. interstimulus interval for dopamine-depleted mice (solid circles). The summary graph from control animals is shown for comparison (same plot as FIG. 1C). b, Activation of CB1 receptors inhibits presynaptic neurotransmitter release in dopamine-depleted animals (solid circles). WIN55,212 (1 μM) and AM251 (5 μM) were applied during the time marked by the bars above the graph. Results from reserpine-treated and 6-OHDA treated mice were similar and were pooled together to form the summary graphs in this figure. Error bars are ±SEM.

FIG. 7. Rescue of striatal indirect pathway LTD by URB754. a, URB754 (5 μM) has no effect on EPSC amplitudes (open circles). The bar marks the time of drug application. b, No LTD is present at direct pathway synapses in the presence of URB754 (closed circles). c, URB754 enhances inhibition of EPSCs (open triangles) following a 1-minute application of DHPG (100 μM). d, URB754 rescues indirect pathway LTD in reserpine-treated mice (open triangles). The lack of LTD in reserpine-treated mice (closed circles) is shown for comparison (same plot as FIG. 4a). e, AM251 (1 μM) blocks the URB754-mediated rescue of indirect pathway LTD in reserpine-treated mice (closed triangles). f, URB754 rescues indirect pathway LTD in 6-OHDA-treated mice (open triangles). The lack of LTD in 6-OHDA-treated mice (closed circles) is shown for comparison (same plot as FIG. 4b). Arrows marks the time of LTD induction. Error bars are ±SEM.

FIG. 8. Rescue of motor impairments by URB754. a,b Descent latency in the bar test is shown for reserpine-treated mice (a) or 6-OHDA-treated mice (b) before subsequent drug treatment, and after injection of URB754 (1.5 mg/kg i.p.), quinpirole (1.5 mg/kg i.p.), or URB754 and quinpirole (both at 1.5 mg/kg i.p.). c,d Locomotor activity in the open-field test for animals treated with reserpine (c) or 6-OHDA (d) is plotted as distance traveled (cm) during a 15-minute test period before subsequent drug treatment, and for mice 45-60 minutes after injection of either URB754 (1.5 mg/kg i.p.), quinpirole (1.5 mg/kg i.p.), or URB754 and quinpirole (both at 1.5 mg/kg i.p.). The data for quinpirole alone is the same data shown in FIG. 4e-h, and is displayed here for comparison. Asterisks denote p<0.05 by one-way ANOVA with Tukey's HSD or Dunnett's test. Error bars are ±SEM.

FIG. 9. Schematic of striatal LTD induction. Excitatory axons arising from cortex and thalamus form synapses onto indirect pathway (D2-receptor-expressing) medium spiny neurons (MSNs) (left) and direct pathway (D1-receptor-expressing) MSNs (right). Excitatory synapses at indirect pathway MSNs exhibit higher release probability and larger NMDA currents than direct pathway synapses. Although presynaptic terminals onto both direct and indirect MSNs express cannabinoid CB1 receptors, only indirect pathway MSNs release endocannabinoids in response to mGluR1/5 agonists or high-frequency presynaptic stimulation, most likely by activation of phospholipase Cβ1 signaling. Dopamine, acting via D2 receptors, can enhance endocannabinoid release in response to brief mGluR activation, and is required for tetanus-induced endocannabinoid-mediated LTD.

### DETAILED DESCRIPTION OF THE EMBODIMENTS

The present invention provides methods that utilize regulation of striatal indirect pathway endocannabinoid-mediated long-term depression (eCB-LTD) to improve motor deficits of striatal-based brain disorders. In particular, such methods find use in the treatment of Parkinson's disease. In one embodiment of the invention, a D2 receptor agonist is administered in conjunction with an inhibitor of endocannabinoid degradation. In some embodiments, the inhibitor of endocannabinoid degradation is a fatty acid amine hydrolase inhibitor, or a monoacylglycerol lipase antagonist. The combination provides for a synergistic effect, with improved therapeutic effects. Also provided are kits and systems for practicing the subject methods, as well as methods of use of agents identified in the screening method of the invention.

The subject methods are useful for both prophylactic and therapeutic purposes. Thus, as used herein, the term “treating” is used to refer to both prevention of disease, and treatment of a pre-existing condition. The treatment of ongoing disease, to stabilize or improve the clinical symptoms of the patient, is a particularly important benefit provided by the present invention.

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “an individual” includes one or more individuals and reference to “the method” includes reference to equivalent steps and methods known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Dopamine D2 receptor agonist. As used herein, a dopamine D2 receptor agonist is a compound that selectively or non-selectively activates dopamine D2 receptors. The D2 dopamine receptor is a G protein-coupled receptor located on postsynaptic neurons that is centrally involved in reward-mediating mesocorticolimbic pathways. Signaling through dopamine D2 receptors governs physiologic functions related to locomotion, hormone production, and drug abuse. D2 receptors are also known targets of antipsychotic drugs that are used to treat neuropsychiatric disorders. Somatostatin and dopamine are 2 major neurotransmitter systems that share a number of structural and functional characteristics. Somatostatin receptors and dopamine receptors are colocalized in neuronal subgroups, and somatostatin is involved in modulating dopamine-mediated control of motor activity. The genetic sequence for D2 receptors in a variety of mammals are publicly known and used in the art, e.g. the human cDNA sequence is available in Genbank at accession number M30625 (see Selbie et al. (1989) DNA 8 (9), 683-689, “The major dopamine D2 receptor: molecular analysis of the human D2A”).

Many such agonists are known and used in the art, including, without limitation, (+)-butaclamol (dopamine D2/D3 receptor antagonist); 5-OH-FPPAT (D2/D3 receptor agonist); 7-OH-DPAT (D2/D3 receptor agonist); alpha-dihydroergocryptine; apomorphine (dopamine receptor agonist); aripiprazole; BAM-1110 (D1/D2 dopamine receptor agonist); bromocriptine (selective dopamine D2 receptor agonist); cabergoline (dopamine D2 receptor agonist); domperidone (dopamine D2 antagonist); dopamine; eticlopride (dopamine D2 receptor antagonist); fallypride (dopamine D2/D3 receptor antagonist); lisuride; haloperidol (dopamine D2/D3 receptor antagonist); nemonapride (dopamine D2/D3/D4 receptor antagonist); N-propylnoraporphine (D2 dopamine receptor agonist); pergolide (D1 and D2 receptor agonist); pimozide (dopamine D2 receptor antagonist); quinelorane (dopamine D2/D3 receptor agonist); quinpirole (dopamine D2/D3/D4 receptor agonist); raclopride (dopamine D2/D3 receptor antagonist); ropinirole (dopamine D2/D3/D4 receptor agonist); S32504 (dopamine D2/D3 receptor agonist); sumanirole; spiperone (dopamine D2 receptor antagonist); sulpiride (dopamine D2/D3 receptor antagonist); talipexole (D2 receptor agonist); terguride; U-91356A (dopamine D2 receptor agonist); and the like.

Dopamine D2 receptor agonists are administered at a concentration normally effective as a single agent, or at a concentration less than the normally effective dose.

Inhibitor of endocannabinoid degradation. Endocannabinoids such as anandamide (N-arachidonoylethanolamine) and 2-arachidonoylglycerol (2-AG) are inactivated upon enzymatic hydrolysis. Two of the major enzymes involved in endocannabinoid degradation are the membrane-bound amidase fatty acid amide hydrolase (FAAH); and monoacylglycerol lipase. For the purposes of the present invention, inhibitors may act on either of these enzymes.

Fatty acid hydrolase (FAAH) is a membrane-associated serine hydrolase enriched in brain and liver. FAAH hydrolyzes endogenous bioactive fatty acid amides including as anandamide and oleamide, thus terminating their activity. This enzyme has a broad substrate specificity. A serine residue functioning as a catalytic nucleophile and several other catalytically important residues have been identified in its primary structure.

Since FAAH is recognized as a drug target, a large number of inhibitors have been synthesized and tested (see, for example, Mahadevan and Razdan (2005) AAPS J.7(2):E496-502, herein specifically incorporated by reference). For example, the synthesis and SAR of alkylcarbamic acid aryl esters as FAAH inhibitors has been reported by Tarzia et al and Mor et al. Potent analogs in this class include URB524 and URB597. Arachidonylsulfonyl derivatives have been reported by Segall et al. as novel inhibitors of FAAH with the potency being similar to methyl arachidonyl-fluorophosphonate.

Monoglyceride lipase (MGLL; EC 3.1.1.23) functions together with hormone-sensitive lipase to hydrolyze intracellular triglyceride stores in adipocytes and other cells to fatty acids and glycerol. MGL-degrading enzymatic activity is sensitive to inhibition by sulfhydryl-specific reagents. Inhibition studies of this enzymatic activity by N-ethylmaleimide analogs revealed that analogs with bulky hydrophobic N-substitution were more potent inhibitors than hydrophilic or less bulky agents (see Saario et al. (2005) Chem. Biol. 12(6):649-56, herein specifically incorporated by reference). The substrate analog N-arachidonylmaleimide is reported to be a potent inhibitor. URB754 may be a blocker of monoacylglycerol lipase (MGL).

Inhibitors of endocannabinoid degradation may not demonstrate therapeutic efficacy in the absence of a co-administered dopamine D2 receptor agonist. The inhibitors are thus administered at a dose that is effective in a combined formulation, i.e. at a concentration effective to substantially reduce degradation of endocannabinoids.

As used herein “Parkinson's disease” refers to condition of disturbance of voluntary movement in which muscles become stiff and sluggish, movement becomes clumsy and difficult and uncontrollable rhythmic twitching of groups of muscles produces characteristic shaking or tremor. The condition is believed to be caused by a degeneration of pre-synaptic dopaminergic neurons in the brain. The absence of adequate release of the chemical transmitter dopamine during neuronal activity thereby leads to the Parkinsonian symptomatology.

The term “assessing” includes any form of measurement, and includes determining if an element is present or not. The terms “determining”, “measuring”, “evaluating”, “assessing” and “assaying” are used interchangeably and include quantitative and qualitative determinations. Assessing may be relative or absolute. “Assessing the presence of” includes determining the amount of something present, and/or determining whether it is present or absent. As used herein, the terms “determining,” “measuring,” and “assessing,” and “assaying” are used interchangeably and include both quantitative and qualitative determinations.

The terms “reference” and “control” are used interchangeably to refer to a known value or set of known values against which an observed value may be compared. As used herein, known means that the value represents an understood parameter, e.g., a level of expression of a cytotoxic marker gene in the absence of contact with a transfection agent.

As used herein, “treatment” or “treating” refers to inhibiting the progression of a disease or disorder, e.g., Parkinson's disease, or delaying the onset of a disease or disorder, e.g., Parkinson's disease, whether physically, e.g., stabilization of a discernible symptom, physiologically, e.g., stabilization of a physical parameter, or both. As used herein, the terms “treatment,” “treating,” and the like, refer to obtaining a desired pharmacologic and/or physiologic effect. The effect may be prophylactic in terms of completely or partially preventing a disease or condition, or a symptom thereof and/or may be therapeutic in terms of a partial or complete cure for a disease or disorder and/or adverse affect attributable to the disease or disorder. “Treatment,” as used herein, covers any treatment of a disease or disorder in a mammal, such as a human, and includes: decreasing the risk of death due to the disease; preventing the disease of disorder from occurring in a subject which may be predisposed to the disease but has not yet been diagnosed as having it; inhibiting the disease or disorder, i.e., arresting its development (e.g., reducing the rate of disease progression); and relieving the disease, i.e., causing regression of the disease. Therapeutic benefits of the present invention include, but are not necessarily limited to, reduction of risk of onset or severity of disease or conditions associated with Parkinson's disease.

### Methods of Use

A combined therapy of a D2 receptor agonist in conjunction with an inhibitor of endocannabinoid degradation is administered to a subject suffering from a striatal-based brain disorder. The inhibitor of endocannabinoid degradation may be a fatty acid amine hydrolase inhibitor, or a monoacylglycerol lipase antagonist. Administration may be topical, localized or systemic, depending on the specific disease. The compounds are administered at a combined effective dosage that over a suitable period of time reduces the motor deficits associated with striatal brain disorders, while minimizing any side-effects. It is contemplated that the composition will be obtained and used under the guidance of a physician for in vivo use.

To provide the synergistic effect of a combined therapy, the inhibitors can be delivered together or separately, and simultaneously or at different times within the day. In one embodiment of the invention, a co-formulation is used, where the two components are combined in a single suspension. Alternatively, the two may be separately formulated.

The efficacy of a particular combination and dose of drugs may be determined by in vitro testing, as detailed in the experimental section, or in vivo testing. The dose will vary depending on the specific compounds utilized, patient status, etc., at a dose sufficient to improve patient mobility, while otherwise maintaining patient health.

The active compounds can be incorporated into a variety of formulations for therapeutic administration. Part of the total dose may be administered by different routes. Such administration may use any route that results in systemic absorption, by any one of several known routes, including but not limited to inhalation, i.e. pulmonary aerosol administration; intranasal; sublingually; orally; and by injection, e.g. subcutaneously, intramuscularly, etc.

For injectables, the agents are used in formulations containing cyclodextrin, cremophor, DMSO, ethanol, propylene glycol, solutol, Tween, triglyceride and/or PEG. For oral preparations, the agents are used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and in some embodiments, with diluents, buffering agents, moistening agents, preservatives and flavoring agents.

Formulations are typically provided in a unit dosage form, where the term “unit dosage form,” refers to physically discrete units suitable as unitary dosages for human subjects, each unit containing a predetermined quantity of inhibitor calculated in an amount sufficient to produce the desired effect in association with a pharmaceutically acceptable diluent, carrier or vehicle. The specifications for the unit dosage forms of the present invention depend on the particular complex employed and the effect to be achieved, and the pharmacodynamics associated with each complex in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Depending on the patient and condition being treated and on the administration route, the active compounds are administered in dosages of 0.1 mg to 2000 mg/kg body weight per day, e.g. about 100, 500, 1000, 10,000 mg/day for an average person. Durations of the regimen may be from: 1×, 2× 3× daily. Some of the inhibitors of the invention are more potent than others. Preferred dosages for a given inhibitor are readily determinable by those of skill in the art by a variety of means. A preferred means is to measure the physiological potency of a given compound.

Various methods for administration are employed in the practice of the invention. The dosage of the therapeutic formulation can vary widely, depending upon the nature of the disease, the frequency of administration, the manner of administration, the clearance of the agent from the patient, and the like. The initial dose can be larger, followed by smaller maintenance doses. The dose can be administered as infrequently as weekly or biweekly, or more often fractionated into smaller doses and administered daily, with meals, semi-weekly, and the like, to maintain an effective dosage level.

### Compound Screening

Compound screening may be performed using an in vitro model as described in the examples, where the effectiveness of compounds for the treatment of brain disorders of the striatum is determined by the effect on indirect pathway eCB-LTD in vitro.

Compound screening identifies agents that modulate function of indirect pathway eCB-LTD, and may be further tested in vivo models for Parkinson's disease or other conditions of interest. Of particular interest are screening assays for agents that have a low toxicity for human cells. A wide variety of assays may be used for this purpose. Knowledge of the 3-dimensional structure of the encoded protein, derived from crystallization of purified recombinant protein, could lead to the rational design of small drugs that specifically inhibit activity. These drugs may be directed at specific domains, e.g. the active sites of MGL or FAAH.

The screening methods of the invention may utilize acute or cultures coronal brain slices. For example, acute brain slices may be kept in oxygenated artificial cerebral spinal fluid. Slices may be depleted of dopamine by culture in the presence of, for example, reserpine, or by obtaining brain slices from animals pre-treated with agents to deplete dopamine. Clamp recordings may be taken from MSNs in the dorsolateral striatum, optionally in the presence of an inhibitor of GABA_{A}-mediated currents. Excitatory synaptic currents are evoked by intrastriatal microstimulation. Medium spiny neurons were identified by their morphology and characteristic electrophysiological properties including negative resting membrane potentials and slow capacitance transients.

The cultured brain slices are contacted with a candidate agent, which may be compared to positive and/or negative controls. The LTD at indirect-pathway MSN synapses may be determined, for example as an effect of endocannabinoid activity and/or dopamine activity. In the presence of a candidate drug acting as a D2 receptor agonist the depression of EPSCs in indirect-pathway MSNs is enhanced.

The term “agent” as used herein describes any molecule, e.g. protein or pharmaceutical, with the capability of altering or mimicking the physiological function of a TBT polypeptide. Generally a plurality of assay mixtures are run in parallel with different agent concentrations to obtain a differential response to the various concentrations. Typically one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Test agents can be obtained from libraries, such as natural product libraries or combinatorial libraries, for example. A number of different types of combinatorial libraries and methods for preparing such libraries have been described, including for example, PCT publications WO 93/06121, WO 95/12608, WO 95/35503, WO 94/08051 and WO 95/30642, each of which is incorporated herein by reference.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

Compounds that are initially identified by any of the foregoing screening methods can be further tested to validate the apparent activity. The basic format of such methods involves administering a lead compound identified during an initial screen to an animal that serves as a model for humans and then determining efficacy in treatment of the targeted condition. For compounds identified herein, a combination assay may be performed in vivo, where a candidate compound is co-administered with either a dopamine D2 receptor agonist or an antagonist of endocannabinoid degradation, as appropriate. The animal models utilized in validation studies generally are mammals. Specific examples of suitable animals include, but are not limited to, primates, mice, and rats.

Animal models of interest include animals where dopamine has been depleted, e.g. after administration of reserpine; 6-Hydroxydopamine, etc. Measurements of movement may be made following administration of candidate agents, e.g. a bar test for catalepsy; descent latency time; horizontal locomotor activity; and the like, as known in the art.

Active test agents identified by the screening methods can serve as lead compounds for the synthesis of analog compounds. Typically, the analog compounds are synthesized to have an electronic configuration and a molecular conformation similar to that of the lead compound. Identification of analog compounds can be performed through use of techniques such as self-consistent field (SCF) analysis, configuration interaction (CI) analysis, and normal mode dynamics analysis. Computer programs for implementing these techniques are available. See, e.g., Rein et al., (1989) Computer-Assisted Modeling of Receptor-Ligand Interactions (Alan Liss, New York).

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

### EXPERIMENTAL

### Endocannabinoid-Mediated Rescue of Striatal LTD and Motor Deficits in Models of Parkinson's Disease

The striatum is a major forebrain nucleus that integrates cortical and thalamic afferents and forms the input nucleus of the basal ganglia. Striatal projection neurons target the substantia nigra pars reticulata (direct pathway) or the lateral globus pallidus (indirect pathway). Imbalances between neural activity in these two pathways have been proposed to underlie the profound motor deficits observed in Parkinson's disease and Huntington's disease. However, little is known about differences in cellular and synaptic properties in these circuits. Indeed, current hypotheses suggest that these cells express similar forms of synaptic plasticity.

Here we show that excitatory synapses onto indirect-pathway medium spiny neurons (MSNs) exhibit higher release probability and larger N-methyl-D-aspartate receptor currents than direct-pathway synapses. Moreover, indirect-pathway MSNs selectively express endocannabinoid-mediated long-term depression (eCB-LTD), which requires dopamine D2 receptor activation. In models of Parkinson's disease, indirect-pathway eCB-LTD is absent but is rescued by a D2 receptor agonist or inhibitors of endocannabinoid degradation. Administration of these drugs together in vivo reduces parkinsonian motor deficits, suggesting that endocannabinoid mediated depression of indirect-pathway synapses has a critical role in the control of movement. These findings have implications for understanding the normal functions of the basal ganglia, and provide an approach treatment of striatal-based brain disorders.

Experimental and clinical work based on anatomical and molecular differences in indirect-pathway and direct-pathway MSNs has led to the suggestion that these pathways contribute differentially to the pathophysiology of striatal disorders. However, progress has been hampered by a lack of knowledge about possible physiological differences between direct-pathway and indirect-pathway MSNs.

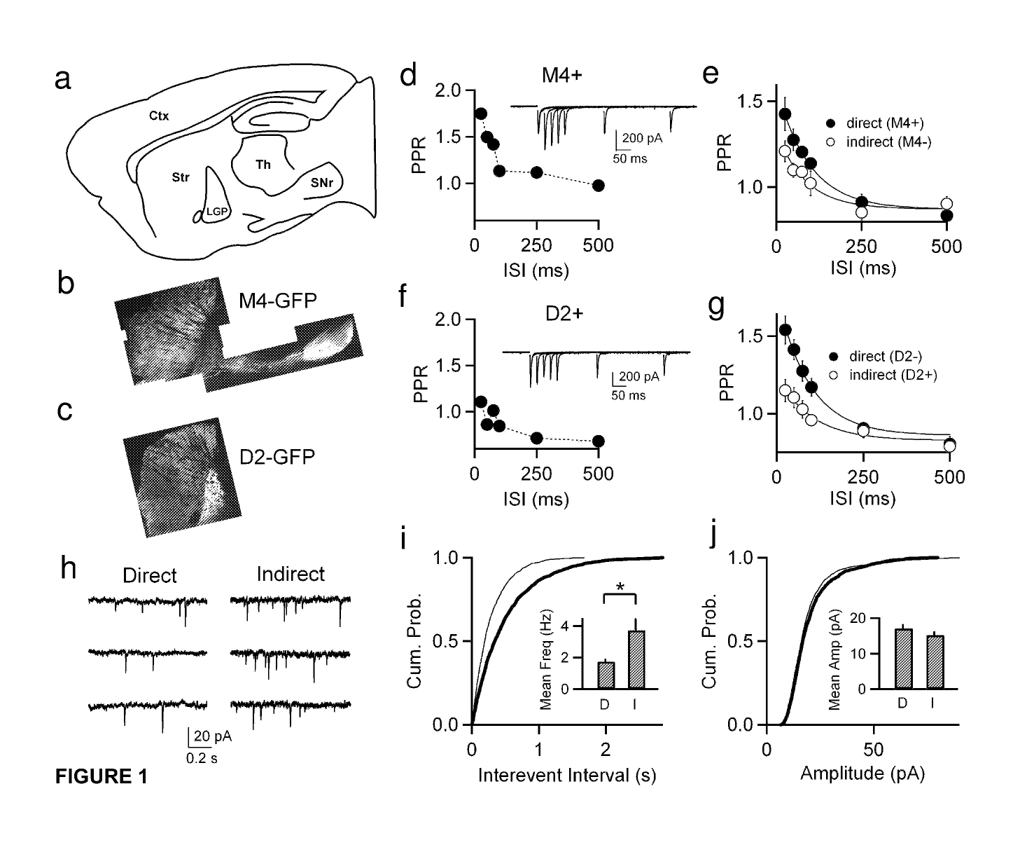

To compare the cellular and synaptic properties of these two classes of MSN we took advantage of bacterial artificial chromosome (BAC) transgenic mice that confer cell-type-specific expression of green fluorescent protein (GFP) in distinct neuronal subpopulations, including MSNs of the direct and indirect pathways. Expression of GFP from the muscarinic M4 receptor locus labelled striatonigral MSNs of the direct pathway (M4-GFP; FIG. 1b). In contrast, GFP expression driven from the dopamine D2 receptor locus labelled striatopallidal MSNs of the indirect pathway (D2-GFP, FIG. 1c).

Whole-cell voltage-clamp recordings from GFP-positive and GFP-negative MSNs in slices from the M4-GFP line revealed that synapses onto direct-pathway MSNs had larger paired-pulse ratios (PPRs) than synapses onto indirect-pathway MSNs (direct-pathway PPRs at 50 ms, 1.28±0.06, n=11; indirect-pathway PPRs at 50 ms, 1.1±0.04, n=7; P, 0.05; FIG. 1d, e). This suggests that indirect pathway synapses have a higher probability of neurotransmitter release than direct-pathway synapses.

To confirm that the M4-GFP and D2-GFP mouse lines label complementary populations of MSNs, we conducted similar experiments in the D2-GFP mouse line. Consistent with our results was our observation that excitatory synapses on direct-pathway MSNs (now GFP negative) in the D2-GFP mouse line had larger PPRs than indirect-pathway (now GFP positive) synapses (direct-pathway PPRs at 50 ms, 1.41±0.06, n=10; indirect-pathway PPRs at 50 ms, 1.1±0.07, n=9; P>0.05; FIG. 1f, g). To examine synaptic properties further in these two cell populations, we recorded miniature excitatory postsynaptic currents (mEPSCs) (FIG. 1h). Direct-pathway neurons had a lower mEPSC frequency than indirect-pathway neurons (direct, 1.7±0.1 Hz, n=4; indirect, 3.7, 0.7 Hz, n=5; P<0.05; FIG. 1i), whereas no differences were observed in mEPSC amplitudes across the two populations (direct, 17.1±1 pA, n=4; indirect, 15.1±0.9 pA, n=5; P>0.05; FIG. 1j). These results provide further evidence that synapses on indirect-pathway MSNs have a higher probability of neurotransmitter release than synapses on direct-pathway MSNs.

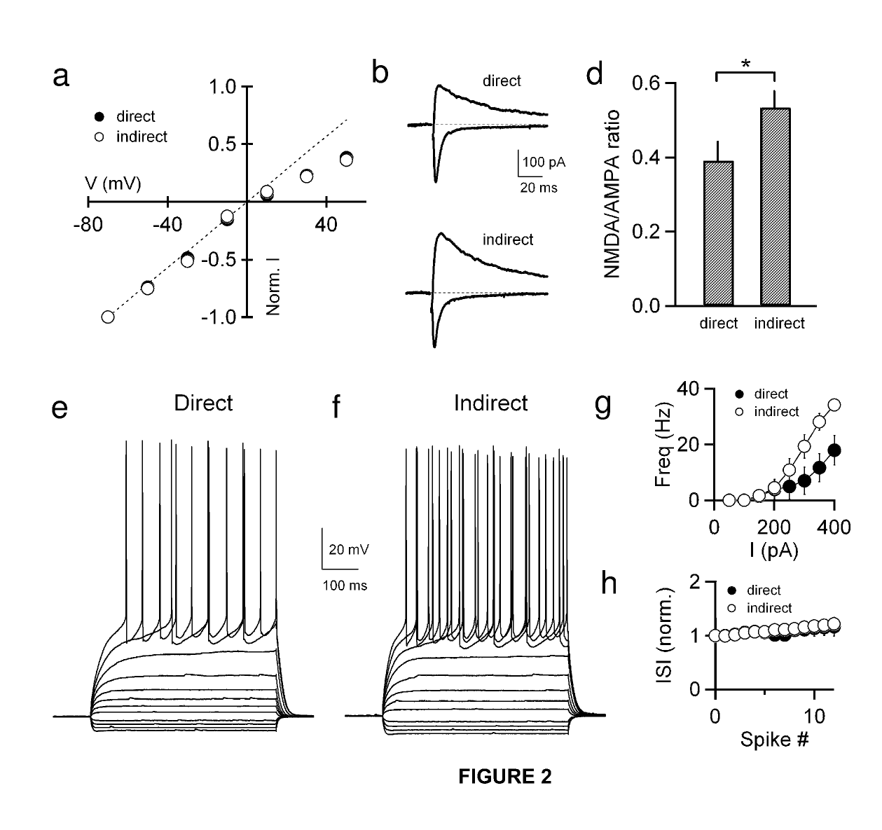

The lack of differences in mEPSC amplitude suggests that the α-amino-3-hydroxy-5-methyl-4-isoxazole propionic acid (AMPA) receptor densities at synapses on the two populations of MSNs are similar. To determine whether the subunit composition of synaptic AMPA receptors might differ, we recorded EPSCs at a range of membrane potentials. In both direct-pathway and indirect-pathway MSNs, EPSCs showed moderate inward rectification (current ratio between +50 mV and −50 mV: direct, 0.51±0.07, n=4; indirect, 0.48±0.03, n=5; FIG. 2a) as well as similar decay time constants (direct, 5.2±0.1 ms, n=18; indirect, 5.4±0.1 ms, n=15), probably indicating a mixture of GluR2-containing and GluR2-lacking AMPA receptors in both MSN populations.

To examine possible differences in synaptic N-methyl-D-aspartate (NMDA) receptors, we measured the ratio of NMDA receptor-mediated synaptic currents (current at 50 ms, V_{hold}=+40 mV) to AMPA receptor-mediated synaptic currents (peak current, V_{hold}=−60 mV). Indirect-pathway synapses had significantly larger NMDA/AMPA ratios than direct-pathway synapses (direct, 0.39±0.05, n=14; indirect, 0.53±0.04, n=14; P<0.05; FIG. 2b-d), although the decay time constants of NMDA receptor-mediated EPSCs were similar in both populations (direct, 51.3±0.5 ms, n=15; indirect, 49.5±0.7 ms, n=14). These findings suggest that indirect-pathway synapses have a higher density of NMDA receptors than direct-pathway synapses but the subunit compositions of synaptic NMDA receptors in the two populations are similar.

We next examined whether the intrinsic membrane excitability of direct-pathway and indirect-pathway MSNs differed. Although the resting membrane potentials and input resistances of the two populations were similar (direct, −91±1 mV, 51±8 MΩ, n=7; indirect, 91±1 mV, 64±8 MΩ, n=8; P>0.05 for both measures) indirect-pathway MSNs fired at nearly twice the rate of direct-pathway neurons in response to depolarizing current injection (FIG. 2e-g). No adaptation in the frequency of spiking during the depolarization was observed in either population (FIG. 2h).

These electrophysiological differences between indirect-pathway and direct-pathway MSNs may be important for spike integration, downstate to upstate transitions, and the induction of synaptic plasticity. For example, the higher probability of release at indirect pathway synapses might enhance the activation of metabotropic glutamate receptors (mGluRs), whereas the increased excitability of indirect-pathway MSNs may increase the activation of L-type calcium channels. Indeed, the most prominent form of synaptic plasticity in the striatum is LTD, which requires the activation of L-type calcium channels and mGluRs, leading to the release of endocannabinoids and a long-lasting inhibition of neurotransmitter release. That striatal LTD also requires D2 receptor activation provides further support for the idea that it may be more robust at indirect-pathway MSNs. We therefore examined the properties of LTD at direct-pathway and indirect-pathway synapses.

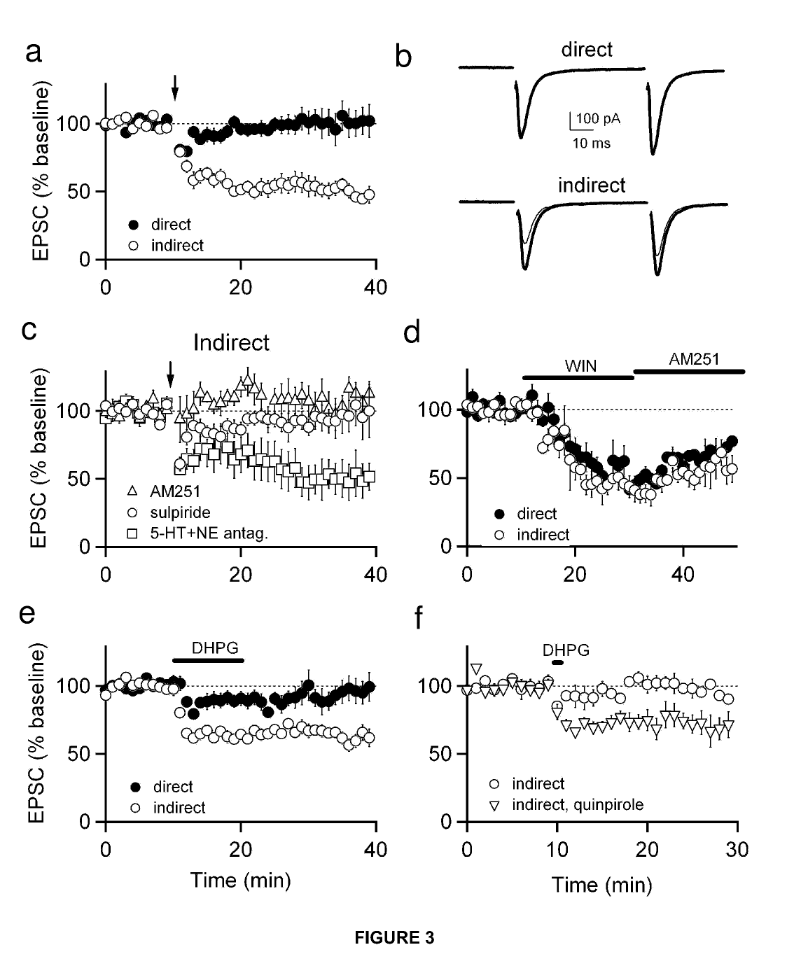

High-frequency afferent stimulation caused robust LTD at indirect-pathway MSN synapses (51±5% of baseline at 30-40 min, n=6, P<0.05; FIG. 3a, b). However, no LTD was apparent at direct pathway synapses (101±10% of baseline at 30-40 min, n=12, P>0.05; FIG. 3a, b) in response to the same induction protocol. The LTD in indirect-pathway MSNs was blocked by the cannabinoid CB1 receptor antagonist AM251 (1 μM) (107±10% of baseline at 30-40 min, n=5, P>0.05; FIG. 3c), confirming that it was mediated by endocannabinoids. It was also blocked by the D2 receptor antagonist sulpiride (10 μM) (96±12% of baseline at 30-40 min, n=6, P>0.05; FIG. 3c) but not by a battery of serotonin and noradrenaline receptor antagonists (methysergide (4 μM), GR125487 (1 μM), LY278584 (1 μM), prazosin (2 μM) and propranolol (1 μM)) (50±13% of baseline at 30-40 min, n=5, P<0.05; FIG. 3c). These results provide further evidence that endogenous dopamine, but not other monoamines, is critical for the triggering of eCB-LTD. The magnitude of eCB-LTD in indirect-pathway MSNs is nearly twice as large as reported previously, suggesting that in previous studies data from indirect-pathway and direct-pathway MSNs were averaged.

The lack of eCB-LTD in direct-pathway MSN synapses could be due to a lack of presynaptic CB1 receptors on the corresponding presynaptic terminals. However, application of the CB1 receptor agonist WIN55,212 (1 μM) decreased EPSCs at both direct-pathway and indirect-pathway synapses to a similar extent (direct, 56±8% of baseline at 15-20 min after wash-in, n=4, P<0.05; indirect, 47±7% of baseline at 15-20 min after wash-in, n=4, P<0.05; FIG. 3d). This finding suggests that the postsynaptic biosynthesis and/or release of endogenous cannabinoids is different between indirect-pathway and direct-pathway MSNs. To test this hypothesis, we applied the type I mGluR agonist (S)-3,5-dihydroxyphenylglycine (DHPG; 100 μM for 10 min) while holding MSNs at a slightly depolarized membrane potential (+50 mV), a manipulation that elicits endocannabinoid release from MSNs and triggers eCB-LTD. This protocol yielded robust LTD at indirect-pathway synapses (64±5% of baseline at 30-40 min, n=5, P<0.05; FIG. 3e) but only a small and reversible depression at direct-pathway synapses (95±9% of baseline at 30-40 min, n=6, P>0.05). Because dopamine acting through D2 receptors enhances striatal endocannabinoid release and D2 receptor activation is necessary for striatal LTD we next asked whether the D2 receptor agonist quinpirole might enhance the depression of EPSCs in indirect-pathway MSNs in response to a brief (1-min) application of DHPG, neither of which alone is sufficient to elicit LTD. As expected, brief DHPG application alone did not yield a lasting depression of EPSCs (98±5% of baseline at 20-30 min, n=5, P>0.05), but in the presence of quinpirole (10 μM) the same DHPG application generated LTD (73±8% of baseline at 20-30 min, n=6, P<0.05; FIG. 3f).

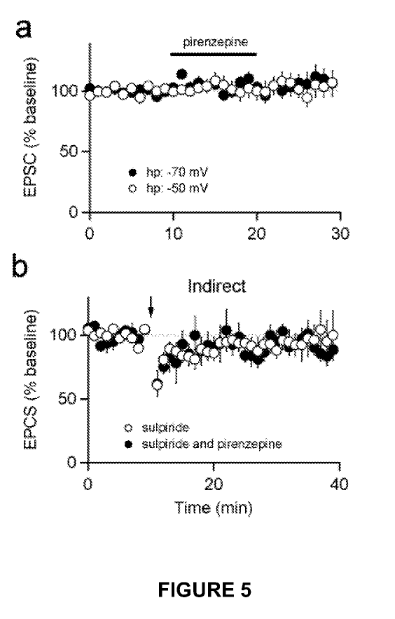

These results show that direct-pathway and indirect-pathway MSNs respond differently to both synaptic stimulation and mGluR activation, suggesting that endocannabinoid release sufficient to trigger eCB-LTD is restricted to indirect-pathway MSNs. The ability to observe LTD at most MSNs in previous study may have been due to differences in experimental protocols that led to a spill-over of endocannabinoids from indirect-pathway postsynaptic neurons onto direct-pathway presynaptic terminals. However, we have not observed such spill-over under a variety of stimulation and recording conditions, nor did we observe a depression of corticostriatal transmission in response to the muscarinic receptor antagonist pirenzepine (FIG. 5).

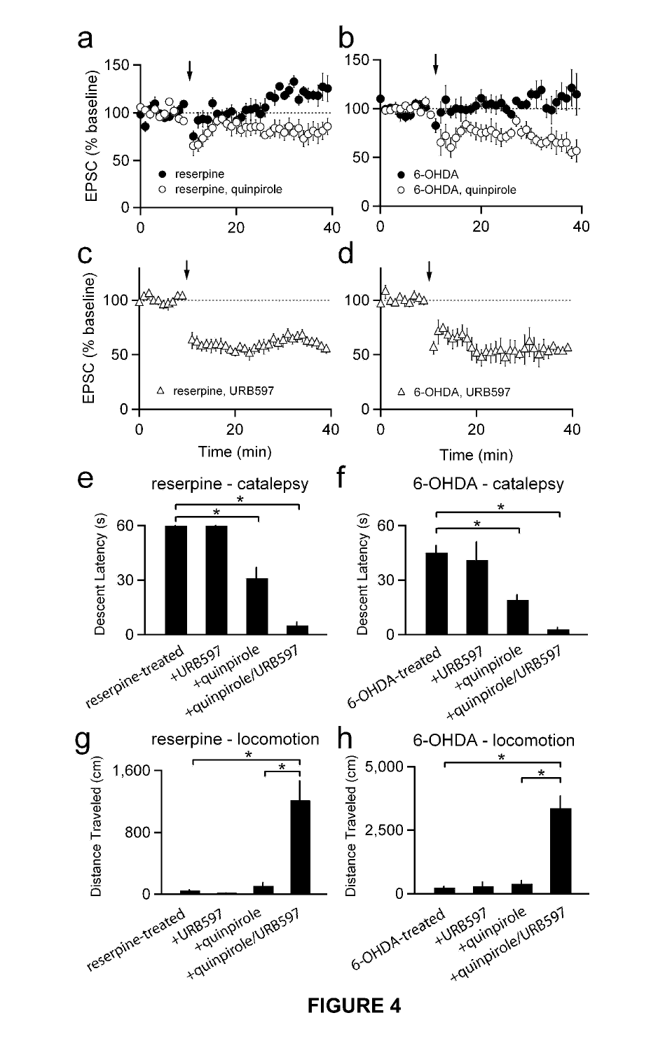

To investigate whether dopamine depletion would block eCBLTD at indirect-pathway MSNs, we used two animal models of Parkinson's disease, treatment with reserpine and with 6-hydroxydopamine (6-OHDA). LTD was not elicited in indirect-pathway MSNs in slices prepared from mice that had received reserpine (5 mg kg⁻¹ intraperitoneally (i.p.)) 18-24 h earlier (122±8% of baseline at 30-40 min, n=6; FIG. 4a), a result providing further evidence that dopamine is critical for triggering eCB-LTD. (Reserpine also decreases the levels of other monoamines, but the data in FIG. 3c show that these monoamines are not required for eCBLTD.) To determine whether eCB-LTD could be rescued in reserpine-treated mice, we applied tetanic stimulation in the presence of quinpirole (10 μM) and found that this facilitated the generation of eCB-LTD at indirect-pathway synapses (81±9% of baseline at 30-40 min, n=5, P<0.05 reserpine-treated versus reserpine-treated with quinpirole; FIG. 4a). We could also rescue LTD by the application of URB597 (1 μM), a potent inhibitor of fatty acid amide hydrolase (FAAH), the degradative enzyme for the endogenous cannabinoid anandamide (62±5% of baseline at 30-40 min, n=5, P<0.05 reserpine-treated versus reserpine-treated with URB597; FIG. 4c). Application of URB597 alone had no effect on EPSC amplitudes (n=4).

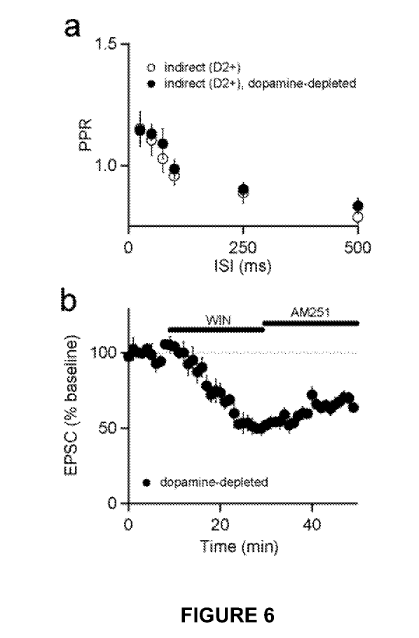

We obtained similar results with mice that had received bilateral 6-OHDA injections into the medial forebrain bundle 48-60 h before slice preparation. Specifically, tetanic stimulation did not elicit LTD at indirect-pathway synapses in slices prepared from 6-OHDA treated mice (111±13% of baseline at 30-40 min, n=7, P>0.05; FIG. 4b) but was rescued by applying either quinpirole (10 μM) (65±8% of baseline at 30-40 min, n=4, P<0.05 6-OHDA-treated versus 6-OHDA-treated with quinpirole; FIG. 4b) or URB597 (1 μM) (55±7% of baseline at 30-40 min, n=4, P<0.05 6-OHDA-treated versus 6-OHDA-treated with URB597; FIG. 4d). Thus, D2 receptor activation or inhibition of endocannabinoid degradation can rescue indirect-pathway eCB-LTD in dopamine-depleted animals. Although biochemical measurements have suggested that endocannabinoid levels in striatal brain tissue may increase after dopamine depletion, we find no physiological evidence consistent with increased endocannabinoid levels in dopamine-depleted animals (FIG. 6).

The success of therapeutic interventions that activate D2 receptors and reduce indirect-pathway activity in Parkinson's disease provided the motivation to test whether inhibiting endocannabinoid degradation, either alone or in combination with D2 receptor activation, could improve the motor deficits in mice treated with reserpine or with 6-OHDA. At 18-24 h after reserpine injections (1 mg kg⁻¹ i.p.), mice displayed pronounced catalepsy (descent latency: more than 60 s, n=14; FIG. 4e) and locomotor activity was minimal (distance traveled in 15 min: 48±11 cm, n=19; FIG. 4g). Similarly, at 48-60 h after bilateral injection of 6-OHDA into the medial forebrain bundle, mice exhibited catalepsy (descent latency: 45±4 s, n=20; FIG. 4f) and minimal open field locomotor activity (distance traveled: 236±52 cm, n=15; FIG. 4h). After administration of URB597 alone (1 mg kg⁻¹ i.p.), no significant decrease in catalepsy or increase in locomotor activity was observed in mice treated with either reserpine or 6-OHDA. Injection of the D2 agonist quinpirole alone (1.5 mg kg⁻¹ i.p.) decreased catalepsy (reserpine-treated mice, descent latency: 31±6 s, n=5, P<0.05; 6-OHDA-treated mice, descent latency: 19±3 s, n=10, P<0.05) but had no significant effect on locomotor activity (reserpine-treated mice, distance traveled: 105±47 cm, n=8, P>0.05; 6-OHDA-treated mice, distance traveled: 385±129 cm, n=5; P>0.05). However, when URB597 was administered together with quinpirole, catalepsy was markedly decreased (reserpine-treated mice, descent latency: 5±2 s, n=7, P<0.05; 6-OHDA-treated mice, descent latency: 3±1 s, n=5, P<0.05) and locomotor activity was increased significantly more than with quinpirole alone (reserpine-treated mice, distance traveled: 1,217±248 cm, n=7, P<0.05; 6-OHDA-treated mice, distance traveled: 3,363±490 cm, n=5, P<0.05). Thus, the effects of these drugs on indirect-pathway eCB-LTD in vitro were predictive of their therapeutic efficacy in two different models of Parkinson's disease.

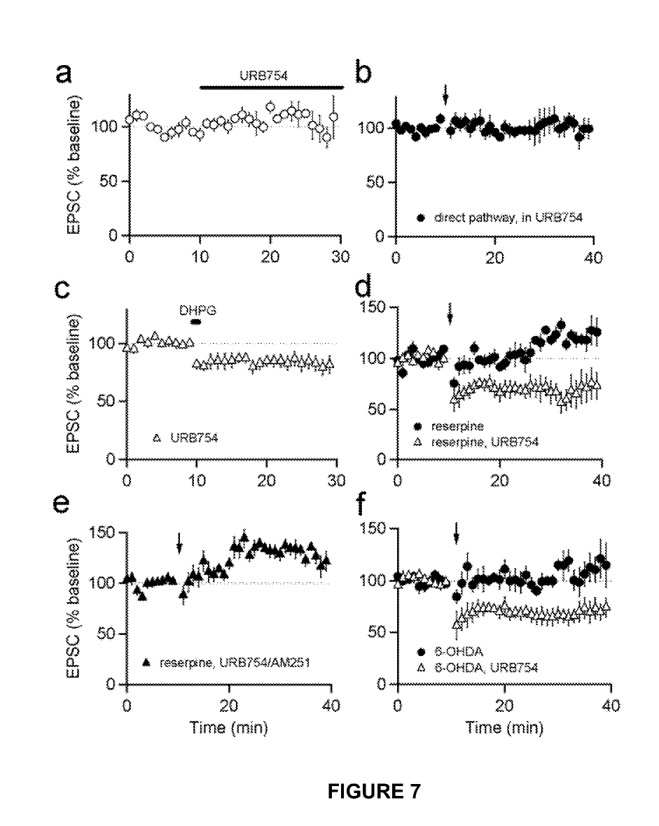

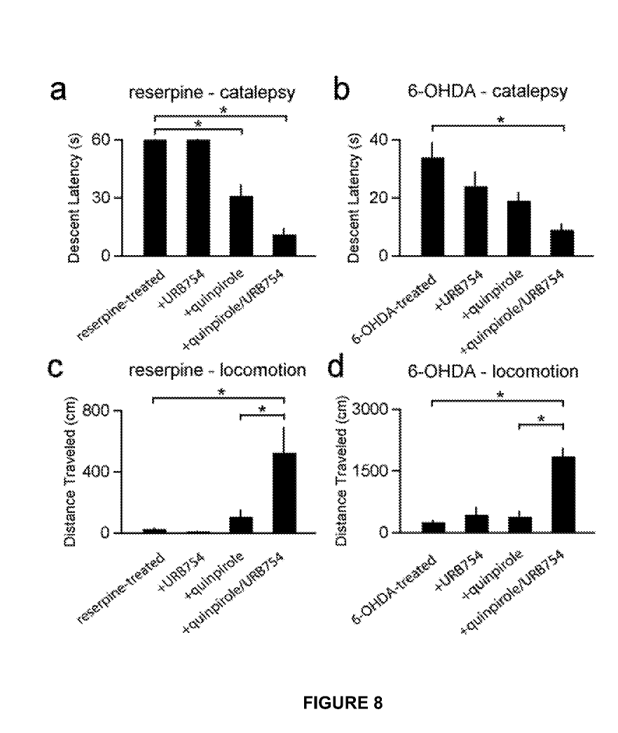

In vivo in the absence of dopamine, endogenous excitatory activity may not generate enough endocannabinoid release from indirect pathway MSNs to yield significant LTD even in the presence of inhibitors of endocannabinoid degradation. However, when endocannabinoids are more effectively released as a result of D2 receptor activation by quinpirole, we propose that the endocannabinoid degradation inhibitor URB597 enhances LTD induction, thereby reducing indirect-pathway neuron activity and more effectively restoring movement. Similar electrophysiological and behavioural results were obtained with URB754 (Makara et al. (2005) Nature Neurosci. 8, 1139-1141) (FIGS. 7 and 8), an inhibitor of the endocannabinoid-degrading enzyme monoacylglycerol lipase.

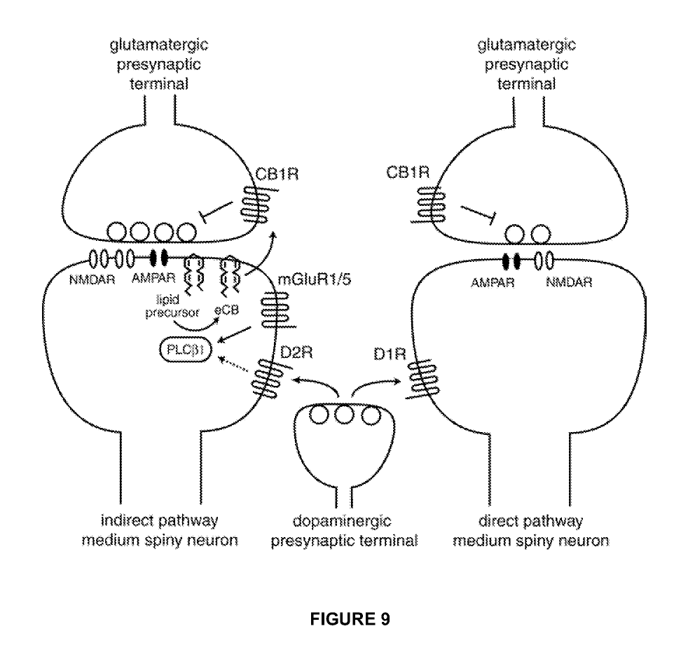

We have found major differences in the cellular and synaptic properties of striatal MSNs in the direct and indirect basal ganglia pathways. Most notably, indirect-pathway MSNs are more excitable and selectively express dopamine-dependent and endocannabinoid dependent LTD (FIG. 9). Dopamine depletion in animal models of Parkinson's disease blocked the generation of eCB-LTD, whereas its rescue by a dopamine D2 receptor agonist or an inhibitor of endocannabinoid degradation predicted the therapeutic benefits of these agents in improving parkinsonian motor deficits in vivo. Consistent with these findings are the observations that both D2 receptor and CB1 receptor knockout mice lack striatal LTD and exhibit profound motor deficits similar to those observed in Parkinson's disease. However, the administration of CB1 receptor antagonists alone does not reduce locomotor activity, and the depletion of dopamine during Parkinson's disease probably results in a host of changes in the cellular and synaptic properties of both direct-pathway and indirect-pathway MSNs. Thus, the elimination of eCB-LTD at indirect-pathway synapses may be one component of the complex pathophysiology of Parkinson's disease.

The ability to generate eCB-LTD in indirect-pathway MSNs but not direct-pathway MSNs seems to be due to differences in the postsynaptic generation of endocannabinoids in response to synaptic activity. Indeed, recent gene profiling of direct-pathway and indirect-pathway MSNs indicates that there are marked differences in the complement of signal transduction molecules and G-protein coupled receptors that they express. The identification of such cell-type-restricted proteins in the striatum along with the characterization of their physiological functions will facilitate the discovery of new drug targets with the therapeutic advantage of allowing specific and independent control of indirect-pathway and direct pathway activity. Together with previous results, our findings specifically demonstrate that manipulation of activity in the indirect basal ganglia pathway by means of modulation of endocannabinoid production will be particularly beneficial for brain disorders that involve dysfunctions of striatal circuitry, such as Parkinson's disease.

### Methods

Electrophysiology. Coronal brain slices (300 μm) were prepared from M4-GFP or D2-GFP heterozygotic BAC-transgenic mice (postnatal days 20-25). Whole cell voltage-clamp and current-clamp recordings were obtained from visually identified GFP-positive or GFP-negative MSNs in dorsolateral striatum at a temperature of 30-32° C., with picrotoxin (50 μM) present to suppress GABA_{A}-mediated currents. Excitatory synaptic currents were evoked by intrastriatal microstimulation with a saline-filled glass pipette placed 50-100 μm dorsolateral of the recorded neuron. All data acquisition and analysis was performed online with custom Igor Pro software.

Dopamine depletion and behaviour. Reserpine was administered i.p. at a concentration of 1 or 5 mgkg⁻¹, and electrophysiological and behavioural experiments were performed 18-24 h after injections. 6-Hydroxydopamine was injected bilaterally into the median forebrain bundle at a concentration of 5 μg μl⁻¹ to a total volume of 2 μL per side, and experiments were performed 48-60 h after injections. Catalepsy was measured with the bar test. Descent latency represents the time in seconds required for the mouse either to remove both paws from a bar (diameter 2 mm) placed 4 cm above the ground, or for one paw to touch the ground. Horizontal locomotor activity was measured in 15-min blocks with the ENV-510 test chamber and Activity Monitor software. Animals were tested 45-60 min after drug administration. All procedures involving animals were approved by the Institutional Animal Care and Use Committee.

Statistics. Summary data are reported as means ±s.e.m. Statistical significance was evaluated with a one-way analysis of variance, with posthoc tests for between-group significance (Tukey's Honestly Significant Difference and Dunnett's test) or the two-tailed unpaired t-test.

Electrophysiology. Coronal slices (300 μM thick) containing the dorsal striatum were prepared from the brains of 20- to 25-day-old M4-GFP or D2-GFP BAC-transgenic mice. D2-GFP and M4-GFP FVB founder mice generated by the GENSAT project were backcrossed into C57/BL6 mice for >5 generations, and all experiments were performed in heterozygotes. Because the electrophysiological results from the two BAC-transgenic mouse lines were very similar, we grouped data from both lines and present it as either direct or indirect pathway. Slices were superfused with an external saline solution containing (in mM): 125 NaCl, 2.5 KCl (or 4.5 KCl where noted), 2 CaCl₂, 1 MgCl₂, 26 NaHCO₃, 1.25 NaH₂PO₄, and 12.5 glucose, bubbled with 95% O₂/5% CO₂. Picrotoxin (50 μM) was added to the external solution to suppress synaptic currents mediated by GABA_{A} receptors. WIN55, 212-2, AM251, DHPG, sulpiride, and quinpirole were purchased from Tocris; URB597 and URB754 were purchased from Cayman Chemical, and carbachol was purchased from Sigma-Aldrich. Drugs were made up as stock solutions and added to the perfusing solution at their final concentrations immediately before application. All recordings were performed at a temperature of 30-32° C.

Whole-cell voltage- and current-clamp recordings from medium spiny neurons were obtained using IR-DIC video microscopy as described previously (Kreitzer & Malenka (2005) Journal of Neuroscience 25, 10537-45). Medium spiny neurons were identified by their morphology and characteristic electrophysiological properties including negative resting membrane potentials and slow capacitance transients. For voltage-clamp recordings, glass electrodes (2.5-3 MΩ) were filled with a solution containing (in mM): 120 CsMeSO₃, 15 CsCl, 8 NaCl, 0.2 EGTA, 10 HEPES, 2 Mg-ATP, 0.3 Na-GTP, 10 TEA, 5 QX-314, adjusted to pH 7.3 with CsOH. For experiments measuring the rectification of AMPA currents, 0.1 mM spermine was included in the pipette. For current-clamp recordings, electrodes were filled with a solution containing (in mM): 130 KMeSO₃, 10 NaCl, 2 MgCl₂, 0.16 CaCl₂, 0.5 EGTA, 10 HEPES, adjusted to pH 7.3 with KOH. Resting membrane potential measurements were performed immediately after break-in. The liquid junction potential, measured at 7 mV, was corrected. Synaptic currents were monitored at a holding potential of −70 mV or −50 mV as stated in the text. Miniature EPSCs were recorded at −70 mV in 1 μM tetrodotoxin. Access resistance and leak currents were monitored continuously and experiments were rejected if these parameters changed by more than 15% during recording.

Excitatory medium spiny neuron afferents were stimulated with a glass electrode filled with external saline and placed between the recorded medium spiny neuron and cortex, typically ˜50-100 μm from the cell body. In some experiments (see below), a bipolar twisted tungsten electrode was placed in the deep cortical layers near the border of the corpus callosum. Stimulus intensity was adjusted to yield EPSC amplitudes between 200-400 pA. Intracortical stimulation was used during pirenzipine application experiments (n=5 of 13) and when attempting to elicit direct pathway LTD (n=4 of 12). Voltage-clamp recordings were performed using an Axopatch 200B (Axon Instruments), filtered at 2 kHz and digitized at 10 kHz. Current-clamp recordings were performed using an Axopatch 1D (Axon instruments), filtered at 5 kHz and digitized at 10 kHz.

Acquisition and analysis were performed using custom Igor Pro software. Paired-pulse ratios are defined as EPSC₂/EPSC₁. NMDA/AMPA ratios were calculated as the ratio of the magnitude of the EPSC at +40 mV at 50 ms following stimulation (NMDA) to the peak of the EPSC at −60 mV (AMPA). For current-clamp experiments, firing frequency was analyzed as the number of spikes occurring during the current injection. Analysis of interspike intervals was only performed on trials in which neurons fired >10 Hz. The magnitude of LTD was calculated by averaging EPSC values from 20 to 30 minutes following the induction protocol and comparing this to the average EPSC during the 10 minute baseline. Summary data are reported as mean ±SEM. For each set of manipulations that are presented in the figures, control experiments were interleaved with the experimental manipulation and then combined to generate the control summary graphs. Statistical significance was evaluated using either one-way ANOVA with posthoc tests for between-group significance (Tukey HSD and Dunnett's test) or using the two-tailed unpaired t test.

Confocal Microscopy. A Zeiss LSM 510 confocal imaging system was used in conjunction with the Zeiss Axiovert 100 M microscope to acquire images from saline-perfused, sagittal brain slices of either M4-GFP or D2-GFP BAC-transgenic mouse lines. A Zeiss 10× oil-immersion objective was used in conjunction with standard fluorescein filter sets and 488 nm laser excitation.

Reserpine treatment. To study the effects of dopamine depletion, reserpine (Sigma-Aldrich) was dissolved in 1% glacial acetic acid at a concentration of 2.5 mg/mL. This solution was diluted with dH₂0 by a factor of 10, to yield a final concentration of 0.1% glacial acetic acid and 250 μg/mL reserpine This reserpine solution was injected i.p. at a concentration of 1 mg/kg or 5 mg/kg as stated in the text. Brain slices from reserpine-treated mice were prepared 18-24 hours after injection, a time when striatal dopamine is <1% of normal levels.

6-hydroxydopamine treatment Bilateral lesions of the substantia nigra pars compacta were achieved by injecting 6-OHDA Bromide (Sigma-Aldrich) into the right and left medial forebrain bundles. Mice were anesthetized with i.p. injections of ketamine (100 mg/kg)/xylazine (10 mg/kg) and placed into a stereotactic frame. 6-OHDA was prepared immediately before injections at a concentration of 5 μg/μL in 0.02% ascorbic acid. Injections were performed using 33 gauge cannulae (Plastics One) attached to a syringe pump (Harvard Apparatus) running at 0.5 μL/min, to a total volume of 2 μL/side at the following coordinates: AP: −1.5 mm; ML±1 mm; DV: 4.8 mm. Brain slices were obtained from 6-OHDA treated animals at 48-60 hour after injections. 6-OHDA-treated animals displayed catalepsy, tremors, and significant hypoactivity and immunoreactivity for tyrosine hydroxylase in the striatum was greatly reduced (34±4% of control, n=4), indicating a significant loss of dopaminergic fibers in the striatum.

Tyrosine hydroxylase staining. The degree of dopamine denervation was assessed at 48 hours post-injection in a subset of 6-OHDA-treated mice by tyrosine hydroxylase (TH) staining. Mouse forebrains were fixed at 4° C. for 1 hour in 4% paraformaldehyde (in PBS), and then kept in PBS overnight at 4° C. Brains were transferred to 30% sucrose (in PBS) for 2 hours, blocked in O.C.T. and frozen. 30 μM coronal slices containing the striatum were collected on slides using a cryostat. Slides were washed (2×5 min) in PBT (PBS and 0.02% Tween 20) and incubated for 15 minutes in blocking solution (0.02% Tween 20, 1% normal goat serum, 0.1% Triton X-100, in PBS). Slides were then incubated for 2 hours at room temperature with TH antibody (1:500 dilution, #P40101-0, Pel-Freez Biologicals) in blocking solution. Slides were again washed (6×5 min) in PBT and incubated for 1.5 hours at room temperature with an Alexa 568-conjugated secondary antibody (goat anti-rabbit, 1:750, Molecular Probes) in blocking solution. Slides were then washed (6×5 min) in PBT and mounted. Images were acquired on a Zeiss Axioskop 2 coupled to a Hamamatsu CCD and analyzed using Metamorph software.

Behavior. 3- to 4-week-old C57/BL6 wild-type mice were injected with reserpine (1 mg/kg i.p.) or 6-OHDA as described above. Due to variability in the effects of these drugs, all mice were assessed prior to behavioral experiments. Following reserpine-treatment, mice that remained alert with eyes fully open and that displayed normal locomotor activity (<5% of mice examined) were not used in experiments. Mice that retained a normal posture, but with pronounced ptosis and catalepsy (˜80% of mice examined) were used for all experiments. Mice with abnormal posture (hind legs extended outward or backward) or that lay on the ground (˜15% of mice examined) were not used. Following 6-OHDA treatment, only mice that displayed significant catalepsy (descent latency >10 seconds) or impairment of movement (distance traveled <600 cm in 15 minutes) were used for experiments (˜80% of mice). Animals were tested 18-24 hours after reserpine injections and 48-60 hours after 6-OHDA injections. Following initial tests, animals were injected with URB597 (1 mg/kg), quinpirole (1.5 mg/kg), or both drugs together. All drugs were injected i.p. in 0.9% NaCl at a volume of 20 μL/gm. The injection solution for URB597 was prepared by diluting a stock solution prepared in DMSO into saline, with a final concentration of 0.5% DMSO. Animals were then tested again at 45-60 minutes following drug injections. URB754 was administered at 1.5 mg/kg i.p. and tested in a similar manner to URB597.

To test catalepsy, a horizontal bar (2 mm diameter) was placed 4 cm above the ground. Mice were grasped by the scruff of the neck and placed so that their forepaws grasped the bar and their hind paws rested stably on the ground. They were held for 2-3 s and then released, at which point a timer was started. Descent latency was measured as the delay to when either both paws were removed from the bar or one paw touched the ground. Trials lasted for a maximum of 60 seconds, and all values represent an average of 3 trials. Locomotor activity in the open-field test was measured using the ENV-510 test environment and Activity Monitor software (Med Associates Inc.). Mice were placed inside an 11×11 inch box with three 16-beam IR arrays, and distance traveled (cm) was calculated for 15-minute time blocks. All procedures involving animals were in accordance with institutional guidelines (IACUC approved).

Endocannabinoid release is not regulated by muscarinic receptors. To test whether inhibition of muscarinic M1 receptors by the specific antagonist pirenzepine depresses excitatory synaptic transmission in striatal slices via enhancement of endocannabinoid release, we applied pirenzepine (10 μM) while recording EPSCs. This manipulation had no effect on EPSC amplitudes (FIG. 5a) (n=13; 104±4% of baseline at 5-10 minutes after wash-in) despite: (1) holding some cells at −50 mV to better activate L-type calcium channel signaling (n=5), (2) increasing extracellular potassium (to 4.5 mM) to enhance cholinergic tone (n=7), and (3) stimulating in cortex, to specifically activate corticostriatal fibers (n=5). We also tested whether M1 receptor inhibition by pirenzepine could rescue LTD in the presence of sulpiride. However, LTD was not elicited in the presence of sulpiride and pirenzepine (FIG. 4b) (93±5% of baseline at 30-40 minutes, n=4). These results are inconsistent with the hypothesis that acetylcholine release from cholinergic interneurons in striatal slices regulates endocannabinoid release and eCB-LTD. Instead, our data suggest that D2 receptor regulation of eCB-LTD is occurring in indirect pathway MSNs.

Dopamine depletion and basal synaptic transmission. Biochemical measurements suggest that endocannabinoid levels in striatal brain tissue may be increased following dopamine-depletion using either reserpine or 6-OHDA. However, we find no physiological evidence of increased endocannabinoid levels in dopamine-depleted animals. The pharmacological rescue of LTD in dopamine-depleted mice indicates that eCB-LTD has not been occluded by elevations in endocannabinoid levels. Paired-pulse facilitation at indirect pathway synapses is also unchanged after reserpine or 6-OHDA treatment (FIG. 5a) (control PPR with 50 ms interstimulus interval: 1.1±0.04, n=7; PPR after dopamine-depletion with 50 ms interstimulus interval: 1.1±0.02, n=13, p>0.05 control vs. dopamine-depleted), consistent with previous results. Moreover, the magnitude of presynaptic modulation by the CB1 agonist WIN55,212 (1 μM) is normal in dopamine-depleted animals, also indicating that no occlusion of presynaptic inhibition by endocannabinoids has occurred (FIG. 5b) (58±5% of baseline at 15-20 min after washing, n=4, p>0.05 control vs. dopamine-depleted). Together, these results suggest that if endocannabinoid levels are increased in striatal tissue from dopamine-depleted animals, these levels do not have any physiological effect on neurotransmitter release probability or LTD induction.

Pharmacological rescue of LTD and motor deficits by URB754. We also tested the efficacy of a second inhibitor of endocannabinoid degradation—URB754, a putative blocker of monoacylglycerol lipase (MGL), the degradative enzyme for the endocannabinoid 2-arachidonoyl glycerol (2-AG). URB754 (5 μM) had no effect on synaptic transmission when washed in alone (FIG. 7a), nor did it unmask LTD at direct pathway neurons (FIG. 7b). However, it did enhance endocannabinoid-mediated inhibition following brief DHPG applications (FIG. S3c). In the presence of URB754, a brief 1-minute DHPG application that alone was not sufficient to elicit LTD (see FIG. 3e), gave rise to small but significant LTD (84±6% of baseline at 20-30 min, n=7, p<0.05). Analogous to our experiments with URB597 (See FIG. 4), we used reserpine- and 6-OHDA-treated mice to investigate whether enhancing endocannabinoid signaling by inhibiting endocannabinoid degradation could rescue indirect pathway LTD. Reserpine-treated animals did not display eCB-LTD at indirect pathway synapses (FIG. 7d, closed circles) (same data as FIG. 4a, closed circles, shown for comparison). In reserpine-treated animals, we were able to rescue LTD by application of URB754 (5 μM) (FIG. 7d) (68±13% of baseline at 30-40 min, n=6, p<0.05 reserpine-treated vs. reserpine-treated with URB754) and this LTD was blocked by addition of the CB1 antagonist AM251 (1 μM) (FIG. 7e) (129±7% of baseline at 30-40 min, n=4), confirming that it requires endocannabinoid signaling. We obtained similar results using bilateral 6-OHDA-treated animals. 6-OHDA-treated animals did not display LTD (FIG. 7f, closed circles) (same data as FIG. 4b, closed circles, shown for comparison). We were able to rescue indirect pathway LTD in 6-OHDA-treated mice by including URB754 in the external saline (5 μM) (FIG. 7f) (69±9% of baseline at 30-40 min, n=4, p<0.05 6-OHDA-treated vs. 6-OHDA-treated with URB754). We also tested whether inhibiting endocannabinoid degradation with URB754, either alone, or in combination with D2 receptor activation, could improve Parkinsonian motor deficits in reserpine- and 6-OHDA-treated mice (FIG. 8). Following systemic administration of the MGL inhibitor URB754 alone (1.5 mg/kg i.p.), no significant decrease in catalepsy or increase in locomotor activity was observed in either reserpine- or 6-OHDA-treated mice. Injection of the D2 agonist quinpirole alone (1.5 mg/kg i.p.) decreased catalepsy (reserpine-treated mice, descent latency: 31±6 seconds, n=5, p<0.05; 6-OHDA-treated mice, descent latency: 19±3 seconds, n=10, p<0.05), but had no significant effect on locomotor activity (reserpine-treated mice, distance traveled: 105±47 cm, n=8, p>0.05; 6-OHDA-treated mice, distance traveled: 385±129 cm, n=5; p>0.05) (quinpirole data in FIG. 8 same as FIG. 4, shown for comparison). However, when URB754 was coadministered with quinpirole, catalepsy was significantly decreased (reserpine-treated mice, descent latency: 11±3 seconds, n=5, p<0.05; 6-OHDA-treated mice, descent latency: 9±3 seconds, n=6, p<0.05), and locomotor activity was increased significantly more than with quinpirole alone (reserpine-treated mice, distance traveled: 525±164 cm, n=10, p<0.05; 6-OHDA-treated mice, distance traveled: 1849±205 cm, n=5, p<0.05). Thus, the actions of the putative MGL inhibitor URB754 were very similar to the actions of the FAAH inhibitor URB597, indicating that both anandamide and 2-AG are likely released from striatal MSNs and play an important role in synaptic plasticity and behavior.

What is claimed is:

1. A method of treating a subject for Parkinson's disease in a subject, said method comprising:
administering to said subject a combination of drugs, said combination comprising
(a) a D2 receptor agonist; and
(b) an inhibitor of endocannabinoid degradation.

2. The method according to claim 1 wherein said combination provides for a synergistic result.

3. The method according to claim 1, wherein said inhibitor of endocannabinoid degradation inhibits fatty acid amine hydrolase.

4. The method according to claim 1, wherein said inhibitor of endocannabinoid degradation inhibits monoacylglycerol lipase.

5. The method according to claim 1, wherein said subject is an animal model for Parkinson's disease.

6. The method according to claim 1, wherein said subject is a human.

7. A method of identifying compounds for the treatment of brain disorders of the striatum, said method comprising: assessing the effectiveness of a candidate compound on indirect pathway eCB-LTD in vitro.
